(19) United States
(12) Patent Application Publication
(10) Pub. No.: US 2008/0109049 A1
(43) Pub. Date: May 8, 2008

(54) DEVICE & METHOD FOR RELIEVING PAIN

(76) Inventor: Daniel H. Schumann, Laguna Niguel, CA (US)

Correspondence Address: JACKSON, DEMARCO, TIDUS & PACKENPAUGH, 2815 TOWNSGATE ROAD, SUITE 200, WESTLAKE VILLAGE, CA, 91361-3010, US

(21) Appl. No.: 11/692,869

(22) Filed: Mar. 28, 2007

Related U.S. Application Data
(63) Continuation-in-part of application No. 11/594,389, filed on Nov. 8, 2006.

(51) Int. Cl. A61N 1/34

(52) U.S. Cl. 607/46

(57) ABSTRACT

A device and method for relieving pain including a waveform generator electrically interconnected with electrodes wherein methods of operation of the waveform generator include methods relying on patient feedback and conductivity measurements of tissue being subjected to a medical procedure.

### PRIORITY CLAIM

This application is a Continuation-In-Part of and incorporates by reference U.S. Utility patent application No. 11/594,389 filed Nov. 8, 2006.

### FIELD OF THE INVENTION

The present invention relates to devices and methods for relieving pain. In particular, the present invention relates to devices and methods of relieving pain via administration of electrical energy.

### BACKGROUND OF THE INVENTION

Patients who anticipate pain from a medical procedure may avoid the procedure to avoid the pain. Patients considering whether they will submit to a recommended but painful medical treatment will often seek less painful alternative and in cases forgo treatment altogether. And where a painful medical treatment is performed, it is common to manage the associated pain by administering pain relief drugs. But, many of these drugs have systemic effects not limited to the site of the medical procedure and they may also have undesirable side effects such as headache and nausea.

What is needed is a pain relief device and method for desensitizing the site of a medical procedure without adverse side-effects such as are commonly associated with pain relief drugs. The present invention satisfies this and other needs, and provides further related advantages.

### SUMMARY OF THE INVENTION

Now in accordance with the invention, there has been found a simple, effective and relatively inexpensive device and method for desensitizing the site of a medical procedure without adverse side-effects. A drug-free device and method relieves patient discomfort and pain during medical procedures involving penetration of patient tissue, such as the pain caused by one or a series of hypodermic injections. The device utilizes an electrically conductive path conducting an electric current, the path including patient tissue and an electrical conductor other than patient tissue connected in series, the tissue being desensitized by the passage of the electric current to relieve pain associated with a medical procedure involving penetration of patient tissue.

### BRIEF DESCRIPTION OF THE DRAWINGS

FIG. 1 depicts an embodiment in the form of a schematic diagram of the pain relief device of the present invention.

FIG. 2 depicts in the form of a schematic diagram the pain relief device of FIG. 1.

FIG. 3 depicts in the form of a schematic diagram controls associated with the pain relief device of FIG. 1.

FIG. 4 depicts in the form of a schematic diagram a switching function of the pain relief device of FIG. 1.

10 FIG. 5 depicts in the form of a schematic diagram a measurement function of the pain relief device of FIG. 1.

FIG. 6 depicts the pain relief device of FIG. 1.

FIG. 7 depicts electrodes used with the electronic pain relief device of FIG. 1.

FIG. 8 depicts a method of using the pain relief device of FIG. 1,

FIGS. 9a through 9f depict probes for use with the pain relief device of FIG. 1.

### DETAILED DESCRIPTION OF THE PREFERRED EMBODIMENTS

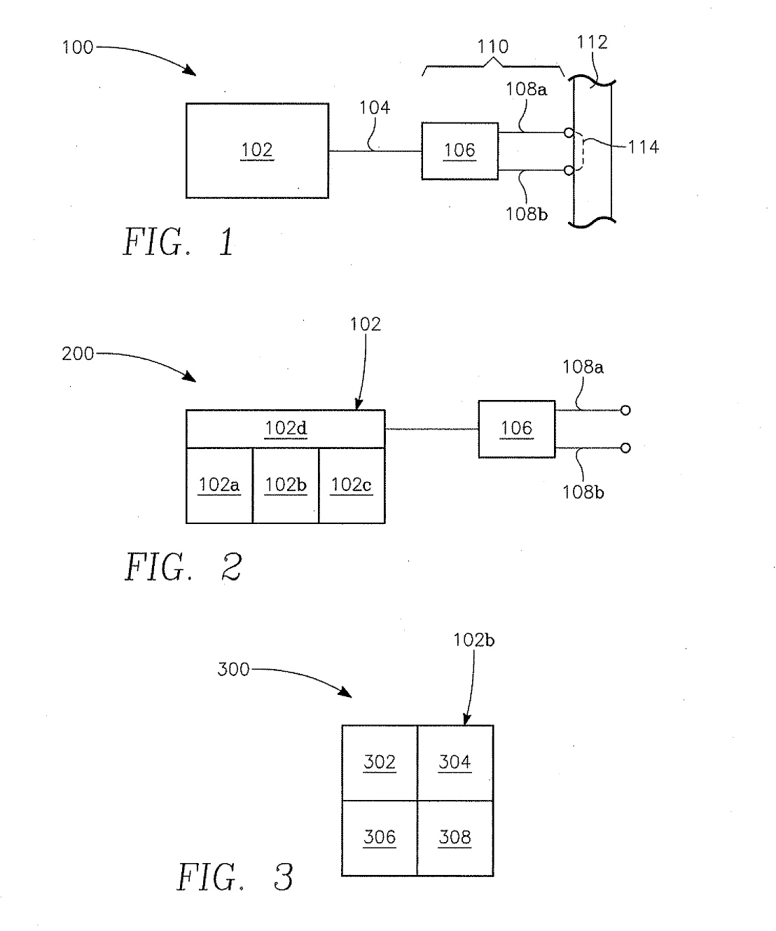

FIG. 1 shows an embodiment of the pain relief device of the present invention 100. Included is a power and controls module 102 electrically interconnected to a probe assembly 110 by one or more electrical conductors 104. The probe assembly includes a probe body 106 and first and second electrodes 108a,b extending from the probe body. Operation of the power and controls module 102 creates a potential difference between the electrodes 108a,b and where an electrical conductor 112 such as human tissue contacts the probes, the potential difference causes a current 114 to flow through the conductive path including the serially interconnected electrodes and conductor or tissue.

FIG. 2 shows an embodiment of the power and controls module 200, Included is a waveform generator 102a for supplying electric power to the electrodes 108a,b, a controller 102b for controlling operation of the waveform generator, a conductivity measurement unit 102c for measuring the conductivity of the conductor 112 and an electrical interconnection section 102d for interconnecting the waveform generator, controller and conductivity measurement unit. In an embodiment, the waveform generator supplies a periodic waveform such as a sinusoidal waveform to the electrodes. And, in some embodiments, the waveform generator supplies a pulse width modulated waveform to the electrodes. In an embodiment, the waveform generator supplies an aperiodic waveform to the electrodes. In various embodiments, any of the waveforms disclosed in U.S. patent application Ser. No. 11/594,389 filed Nov. 8, 2006 may be supplied to the electrodes 108a,b by the waveform generator.

FIG. 3 shows an embodiment of the controller 300. A waveform control section 302 provides for selection of the waveform that will be supplied by the waveform generator, including waveshape, frequency and peak-to-peak amplitude. As used in this specification, waveform includes one or more of wave shape (triangular, square, sine, damped sinusoid and the like), wave frequency and peak-to-peak amplitude of the wave. In a timing control section 304, elapsed time and countdown timers provide means for monitoring the duration of treatment and ending treatment respectively. A man-machine interface section 308 provides for operator control of the pain relief device utilizing one or more display and selection devices. Typical display devices include meters, random addressable displays such as liquid crystal displays and plasma displays, cathode ray tube displays, light emitting diodes and other suitable devices known in the art. Typical selector devices include switches, selector switches, potentiometers, touch sensitive devices including touch sensitive display screens and similar devices known in the art. As will be further described below, automatic control section 306 provides for automatic control of the waveform generator.

The conductivity measurement unit 102c measures the conductivity of a conductor 112 coming into contact with the electrodes 108a,b. In an embodiment, the pain relief device operator utilizes the man-machine interface 308 to initiate and display a conductivity measurement, the conductivity measurement indicating the extent to which tissue between the electrodes has been desensitized by operation of the waveform generator. And in some embodiments, the operator utilizes the man-machine interface to select an automatic mode wherein automated conductivity measurements control the operation of waveform generator.

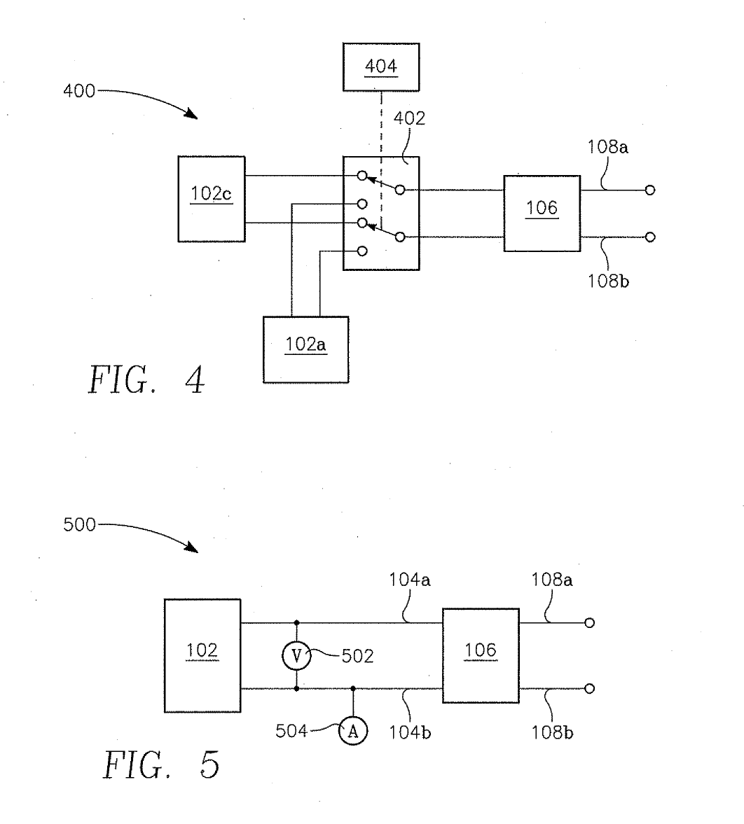

In an embodiment, the electrodes are interconnected with either the waveform generator 102a or the conductivity measurement unit 102c via a switching device which functions as a double-pole-doublethrow switch. This switching function 400 is shown in FIG. 4 where a double pole double throw switch 402 interconnects the electrodes with either of the conductivity measurement unit or the wave form generator. Switching may be manual as with a DPDT switch or automated as with a semiconductor, micro-electromechanical switch (MEM) or other suitable automated switches known in the art 404. In another conductivity measurement embodiment 500 as shown in FIG. 5, the conductivity unit derives conductivity from measurements of the voltage 502 and current 504 supplied to the electrodes 108a,b via conductors 104a,b.

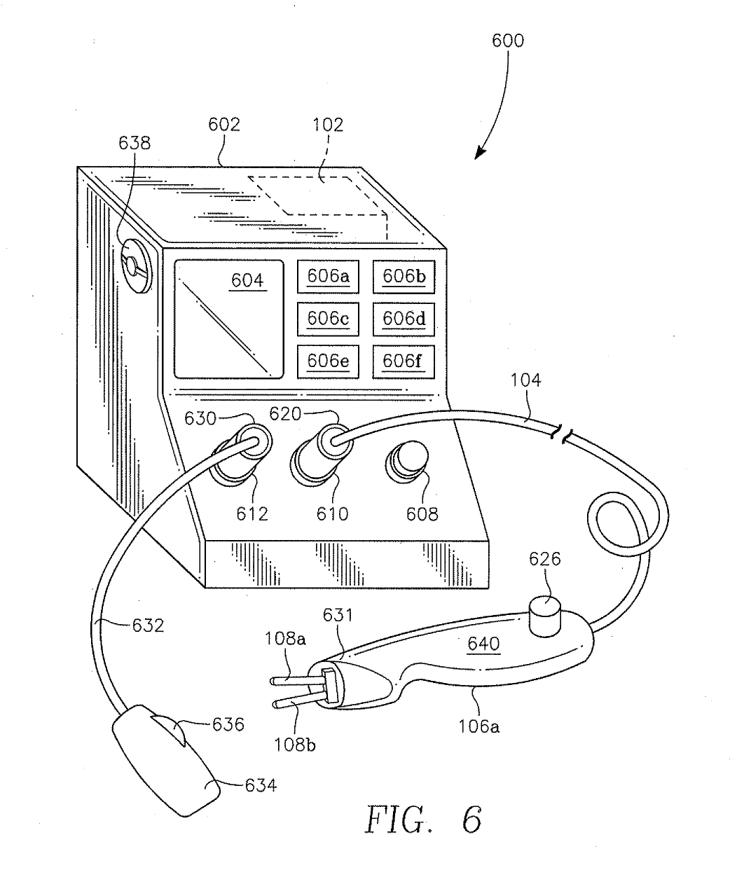

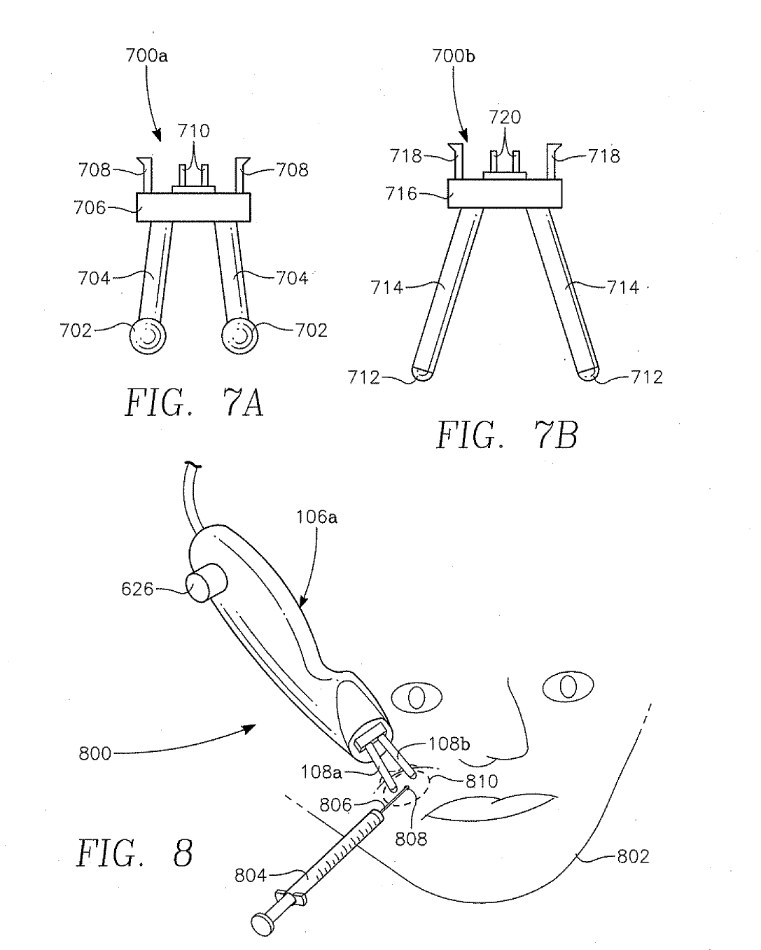

FIG. 6 shows an embodiment of the pain relief device packaged with a separate base unit 600. In this embodiment, the base unit 602 incorporates a power and control module 102 including a waveform generator 102a, a control module 102b with a man-machine interface 308, and a conductivity measuring unit 102c, Connected to the base unit is an elongated probe 106a and an optional waveform control 634.

Included with the man-machine interface of the base unit 308 is a display device 604, six selector controls 606a-f, an off-on switch 608 and an optional audio annunciator 638. Interconnecting the probe with the base unit is an electrical cable 104 having a first plug 620 at one end for mating with a first socket 610 of the base unit. Interconnecting the optional waveform control 634 with the base unit is an electrical cable 632 having a second plug 630 at one end for mating with a second socket 612 of the base unit.

Treatment duration may be monitored by a timer associated with the first selector control 606a. Treatment duration may be limited by a count-down timer associated with the second selector control 606b. The third through fifth selector controls 606c-e adjust the waveform supplied to the electrodes changing frequency, amplitude and wave shape respectively. The sixth selector control 606f initiates a conductivity measurement. Data associated with each of these controls is in various embodiments presented on the display 604.

The probe 106 has an elongated body 640 with two spaced apart electrodes 108a,b at a first end and the interconnecting cable 104 at a generally opposed second end. In an embodiment, the probe includes a waveform amplitude control such as a thumbwheel 626 or similar device for controlling the peak-to-peak voltage of the waveform supplied to the electrodes 108a,b. And, in some embodiments, the frequency of the waveform is controlled by the selector device. In various embodiments, other functions may be controlled by the thumbwheel including selection of a wave shape such as sinusoidal or square wave and turning the pain relief device on and off. In an embodiment, the probe includes a display such as an LCD display for displaying one or more of conductivity, voltage, frequency and wave shape.

In an embodiment, the electrodes 108a,b are detachable from the probe 106. FIG. 7 shows a first detachable electrode assembly 700a and a second detachable electrode assembly 700b. The first detachable electrode assembly has two electrodes, each with a spherical tip 702 joined to an electrode base 706 by an electrode arm 704. Electrical coupling of the first electrode assembly with a probe 106a is accomplished by use of electrode assembly electrical connector 710 which plugs into a mating electrical socket of the probe. Mechanical coupling of the first electrode assembly with the probe is accomplished by use of two electrode assembly mechanical connectors 708 which mate with corresponding mechanical sockets of the probe. In some embodiments, the electrode assembly mechanical connectors also electrically couple the electrodes with the probe, eliminating the need for a separate electrode assembly electrical connector.

The second detachable electrode assembly 700b has two electrodes, each with a hemispherical tip 712 joined to an electrode base 716 by an electrode arm 714. Electrical coupling of the first electrode assembly with a probe 106a is accomplished by use of electrode assembly electrical connector 720 which plugs into a mating electrical socket of the probe. Mechanical coupling of the first electrode assembly with the probe is accomplished by use of two electrode assembly mechanical connectors 718 which mate with corresponding mechanical sockets of the probe. In some embodiments, the electrode assembly mechanical connectors also electrically couple the electrodes with the probe, eliminating the need for a separate electrode assembly electrical connector.

The tips of the electrodes 702, 712 come into contact with patient tissue and conduct the electric power supplied by the waveform generator 102a to the patient tissue. The tips may be made of any conductive material suitable for contact with patient tissue including conductive metals such as stainless steel and conductive composites such as finely divided metal(s) in a ceramic matrix. Each of the electrode tips is electrically interconnected to a respective pin of the electrical connector 710, 720. The electrode assembly base 706, 716 is made from a non-conductive material such as a suitable thermoplastic known in the art. In some embodiments it is desirable to protect against shorting the electrodes. Here, the electrode arms 704, 714 are coated with a non-conductive material such as a polymer coating or they are made from a non-conductive material such as that used for the electrode base.

While elements of the man-machine interface associated with the base unit 602 and the probe 106a are for use by medical personnel operating the pain relief device, the optional waveform control 634 is intended to be operated by the patient. A thumbwheel or similar selector device 636 of the waveform control controls in various embodiments one or more of the peak-to-peak voltage of the waveform, the frequency of the waveform and the wave shape supplied to the electrodes 108a,b.

In operation, the probe 106a is used to desensitize tissue that is penetrated during a medical procedure. Penetration of patient tissue occurs, for example, during hypodermic injections, hypodermic extractions such as the taking of blood samples, incisions, tissue removal such as abrasion procedures, and tissue relocation such as tissue grafts. FIG. 8 shows desensitization for a hypodermic injection 800. Here, a patient 802 is receiving a hypodermic injection into facial tissue. Such injections are typical of those administered during anti-ageing and cosmetic treatments using for example Botox® injections to relax muscles or Restylane® or Juvederm® to provide a dermal filler.

A tissue treatment zone including the underlying tissues 810 is desensitized when the electrodes 108a,b experience a potential difference and the tissues in the treatment zone conduct an electric current responsive to the potential difference. This desensitization reduces the pain resulting from both the puncture wound 808 made in the treatment zone by a hypodermic needle 806 and the subsequent injection of fluid from the syringe 804 into the treatment zone.

In one embodiment, a method of desensitizing a treatment zone without conductivity feedback is used. In a probe placement step, the electrodes 108a,b are placed against the patient's skin so as to “straddle” the injection site 808; the injection may be made anywhere in the treatment zone 810, it need not be made exactly between the electrodes. The probes are placed against the skin with sufficient pressure to ensure good electrical contact and in some embodiments electrical contact is aided by use of a conductive gel that is pre-applied to the probe tips or to the places on the skin where the probe tips will be located. In some embodiments, the pain relief device operator selects a particular waveform to be administered 606b.

In a voltage adjustment step following the probe placement step, the waveform generator 102a is activated and adjusted upward from a peak-to-peak voltage setting of about 0 volts, initiating a flow of electric current through the tissue in the treatment zone 810. Voltage adjustments are made based on feedback from the patient. The voltage level is increased until the patient experiences a gentle “tingling” sensation. The voltage level is further increased until the patient feels a strong, but not painful, tingling sensation. This setting is maintained for a pre-procedure treatment time in the range of 0 to 60 seconds. Where the patient experiences discomfort during this time, the voltage level is reduced accordingly.

In a medical procedure step at the end of the pre-procedure treatment time, the medical procedure begins. During the medical procedure, operation of the waveform generator, resulting in current flows through the treatment zone 810, is maintained and may be adjusted as indicated by the patient. In some embodiments, a remote control 634 is operated by the patient to adjust the electrical output. When the medical procedure is completed, the current flow is maintained for a post-treatment time period in the range of 0 to 60 seconds. Where there are multiple procedures such as multiple injection sites, the electrodes may be slid along the skin for subsequent injections in other nearby locations. Here, the electrical output may be adjusted before moving the electrodes. If there are no further injections, the electrodes are removed from the skin. Or, if there are other injections but the operator elects to move the electrodes rather than sliding them, the electrical output may be adjusted before removing the electrodes from the skin.

In another embodiment, a method of initially desensitizing a treatment zone with conductivity feedback is used. Probe placement is as described above and is followed by an initial conductivity measurement. Voltage adjustment is based on patient feedback as described above but the pre-procedure duration is determined based on reaching a desired conductivity value in the range of about 1.1 to 10 times the initial conductivity measurement. The medical procedure follows as described above. In some embodiments, one or more of an audible 638 or visible 604 indication signals the operator that the desired conductivity has been reached.

In an embodiment, a method of maintaining a treatment zone 810 in a desensitized state during a medical procedure is used. Probe placement is as described above and is followed by an initial conductivity measurement. Voltage adjustment is based on patient feedback as described above and pre-procedure duration is determined by either of the methods described above. The medical procedure follows as described above excepting that voltage adjustments: during the procedure are made automatically 306. Conductivity measured at the end of the pre-procedure time is automatically compared with conductivity measurements made during the procedure. Conductivity decreases signal an automatic increase in the voltage using for example a control algorithm having one or more of proportional, integral and derivative elements. As will be appreciated by those skilled in the art, the capacity to make continuous measurements of conductivity while supplying electric current to the treatment zone (see FIG. 5) facilitates the method of this embodiment.

The pain relief device 100 described above incorporates a power and control module 102 electrically interconnected to a probe assembly 110 Use of the described elongated probe 106a is but one embodiment of the invention. Other probes that employ similar operating means and methods may be used and are encompassed by the present invention.

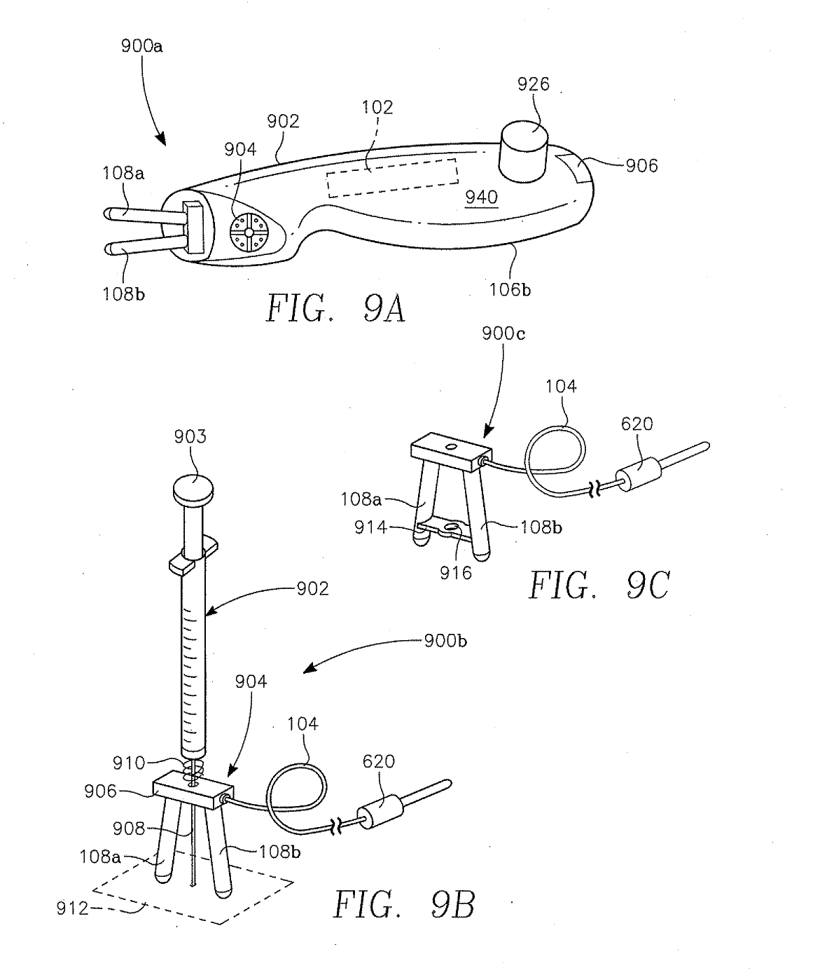

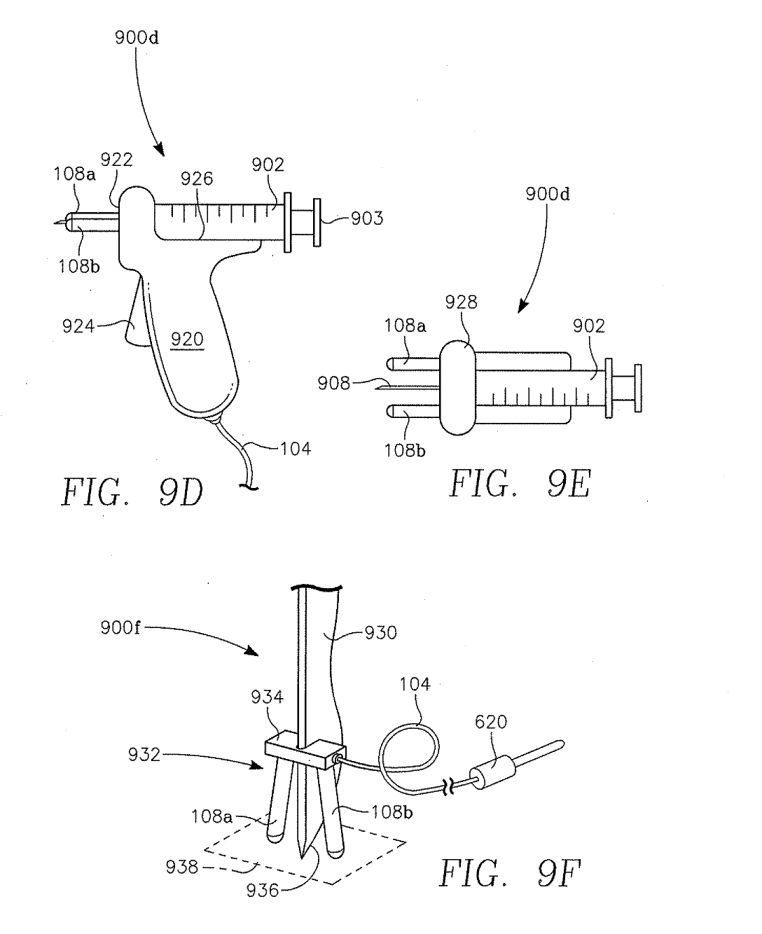

FIG. 9a shows an embodiment of the pain relief device packaged as a stand-alone device 900a. Here, the stand-alone probe 106b has a probe body 940 that incorporates a power and control module 102 having an energy storage element such as a suitable battery known to persons of ordinary skill in the art. In some embodiments, the stand-alone probe uses an external energy source such as that provided by a power cord interconnecting the probe and coupled to a suitable power source such as a mains supply. Power switch 906 turns the device off and on and a selector control 926 varies the voltage supplied to the electrodes 108a,b. In an embodiment a display such as a liquid crystal display 902 provides status information including conductivity and/or time to the operator in some embodiments an audio annunciator 904 signals the operator that a particular conductivity value has been reached.

FIG. 9b shows an embodiment of the probe of the pain relief device received by a hypodermic syringe 900b. A yolk-like probe 904 that is removably coupled to a hypodermic syringe 902 includes two spaced-apart electrodes 108a,b between which the needle 908 of a hypodermic syringe 902 passes. An electrical cable 104 interconnects the electrodes with a base station 602 via a plug 620. In an embodiment, the syringe and the probe are urged apart by a spring 910 located between the syringe and the yolk. In some embodiments, the spring encircles the needle. This probe and syringe configuration provides a combined device that can be held in one hand for both pre-procedure desensitization and the injection when sufficient pressure is applied to collapse the spring and force the needle beyond the plane of the electrode tips 912 and into the tissue of the patient. Depression of the syringe plunger 903 completes the injection.

FIG. 9c shows another embodiment of the yolk-like probe 900c. This probe includes a needle guide 914. The needle guide is a thin elongate member extending between and coupled to each of the electrodes. A hole 916 about midway between the ends of the needle guide receives and guides a needle. In some embodiments, the needle guide hole is dimensioned to indicated the treatment zone 810 that is desensitized by operation of the pain relief device. The needle guide may also be used with the electrodes of the elongated probe 106a,b and in some embodiments has a swivel mount allowing for the angle between the major axes of the syringe and the probe to be varied, preventing interference of the syringe with the probe.

FIGS. 9d-e show an embodiment of the probe of the pain relief device which receives a hypodermic syringe 900d. The probe body 920 is in the shape of a handle having a trigger control 924 for operating the waveform generator 102a and a mount for removably attaching a syringe 902. An electric cable 104 interconnects the electrodes with a base unit 602. In an embodiment, the electrodes extend about perpendicular from an upper vertical face 922 of a generally circular receiver 928 of the handle which receives and holds the needle end of the syringe 902 such that the needle is positioned between the electrodes. An upper horizontal cradle of the handle 926 positioned behind the circular receiver supports the body of the syringe. Following pre-procedure desensitization, the operator pushes the handle toward the patient causing the needle to penetrate the patient's tissue as the electrodes compress their associated springs and recede into the handle. Depression of the syringe plunger 903 completes the injection.

FIG. 9f shows an embodiment of the probe of the pain relief device which is received by a surgical scalpel 900f. An electrode assembly 932 has electrodes 108a,b that lie to either side of a scalpel 930 and an electrical cable 104 interconnects the electrodes with a base unit 602. The electrodes are attached to a bridge 934 which is supported by the scalpel. In an embodiment, the tips of two electrodes 108a,b and the tip of the scalpel blade 936 lie in substantially the same plane. And in some embodiments, the bridge is spring loaded in a manner that urges the electrode tips into a plane below the scalpel blade tip 938. This allows for pre-procedure desensitization of the treatment zone 810 prior to making the incision.

While various embodiments of the present invention have been described above, it should be understood that they have been presented by way of example only, and not limitation It will be apparent to those skilled in the art that various changes in form and details can be made without departing from the spirit and scope of the invention. As such, the breadth and scope of the present invention should not be limited by the above-described exemplary embodiments, but should be defined only in accordance with the following claims and equivalents thereof.

What is claimed is:

1. A device for relieving pain comprising:
an electrically conductive path conducting an electric current; and,
the path including patient tissue and an electrical conductor other than patient tissue connected in series, said tissue being desensitized by the passage of the electric current to relieve pain associated with a medical procedure involving penetration of patient tissue.

2. The device of claim 1 further comprising an electric waveform generator operable to create a potential difference between first and second electrodes in electrical contact with the tissue.

3. The device of claim 2 wherein the duration of operation of the waveform generator prior to penetration of patient tissue is a function of the electrical conductivity of the tissue.

4. The device of claim 3 further comprising an automatic control that adjusts the potential difference between the electrodes during the medical procedure to maintain a pre-determined level of conductivity of the tissue.

5. The device of claim 2 further comprising a patient operated waveform control for controlling the electric waveform supplied to the first and second electrodes.

6. The device of claim 5 wherein the potential difference between the electrodes is controlled by the waveform control.

7. The device of claim 5 wherein the frequency of the waveform supplied to the electrodes is controlled by the waveform control.

8. The device of claim 5 wherein the wave shape of the waveform supplied to the electrodes is controlled by the waveform control.

9. A device for relieving pain caused by use of a hypodermic needle comprising:
a hypodermic needle;
a body bounding a reservoir said reservoir in fluid communication with the needle;
an electrode assembly coupled to the body; and,
the needle passing between first and second electrodes of the electrode assembly.

10. The device of claim 9 further comprising an electric waveform generator operable to create a potential difference between the first and second electrodes.

11. The device of claim 10 wherein the duration of operation of the waveform generator prior to penetration of patient tissue with the needle is a function of the electrical conductivity of the tissue.

12. The device of claim 11 further comprising an automatic control that adjusts the potential difference between the electrodes during the medical procedure to maintain a pre-determined level of conductivity of the tissue.

13. The device of claim 10 further comprising a spring urging the maintenance of a gap between a tip of the needle and a plane, the plane touching a tip of an electrode and about perpendicular to a longitudinal axis of the electrode.

14. The device of claim 10 wherein the first and second electrodes are removably coupled to the body.

15. A device for relieving pain caused by penetrating patient tissue comprising,
first and second spring urged electrodes extending from a probe body, the tips of the electrodes for contacting patient tissue,
an electrical circuit including the electrodes and the tissue therebetween; and,
a electric waveform generator supplying current to the electrical circuit for relieving the pain caused by penetrating patient tissue.

16. The device of claim 15 wherein the duration of operation of the waveform generator prior to penetration of patient tissue is a function of the electrical conductivity of the tissue.

17. The device of claim 16 further comprising an automatic control that adjusts the potential difference between the electrodes during the medical procedure to maintain a pre-determined level of conductivity of the tissue.

18. The device of claim 15 further comprising a handle grip extending from the probe body.

19. The device of claim 18 further comprising a control device coupled to the handle grip for operating the electric waveform generator.

20. The device of claim 18 further comprising a mount operable to removably couple a syringe to the probe body such that a needle of the syringe is about equidistant from each of the electrodes.

21. The device of claim 18 wherein the first and second electrodes are removably attached to the probe body.

22. A device for relieving pain caused by use of a hypodermic needle comprising:
an electrode assembly including first and second spaced apart electrodes said electrodes in electrical contact with patient tissue;
an electric waveform generator operable to create a potential difference between the first and second electrodes; and,
a guide for guiding a hypodermic needle between said electrodes.

23. The device of claim 22 wherein the duration of operation of the waveform generator prior to inserting a needle in patient tissue is a function of the electrical conductivity of the tissue.

24. The device of claim 23 further comprising an automatic control that adjusts the potential difference between the electrodes during the injection to maintain a pre-determined level of conductivity of the tissue.

25. The device of claim 22 wherein the guide for guiding a hypodermic needle extends between and is coupled to each of the first and second electrodes.

26. The device of claim 22 wherein the guide for guiding the hypodermic needle is an arm having an aperture for receiving the needle.

27. The device of claim 26 wherein the aperture is sized to expose tissue desensitized by operation of the device.

28. The device of claim 22 wherein the first and second electrodes are removably attached to the electrode assembly.

29. A device for relieving pain caused by an incision comprising:
a scalpel including a scalpel body, a scalpel blade and an electrode assembly;
the electrode assembly having at least first and second electrodes; and,
the first electrode positioned to contact tissue to one side of the scalpel blade and the second electrode positioned to contact tissue to the opposite side of the scalpel blade.

30. The device of claim 29 further comprising a waveform generator operable to create a potential difference between the first and second electrodes.

31. The device of claim 30 wherein the duration of operation of the waveform generator prior to penetrating patient tissue with the scalpel blade is a function of the electrical conductivity of the tissue.

32. The device of claim 31 further comprising an automatic control that adjusts the potential difference between the electrodes during the inscision to maintain a pre-determined level of conductivity of the tissue.

33. The device of claim 30 wherein the first and second electrodes are removably attached to the electrode assembly.

34. A device for relieving pain comprising:
a waveform generator supplying electric current to an electrically conductive path including two electrodes, said electrodes for making electrical contact with patient tissue;
the tissue being desensitized by the passage of the electric current for relieving pain associated with a medical procedure involving penetration of patient tissue;
the duration of operation of the waveform generator prior to penetration of patient tissue being a function of the electrical conductivity of the tissue; and,
the potential difference between the electrodes during the medical procedure being automatically adjusted to maintain a pre-determined level of conductivity of the tissue.

35. The device of claim 34 further comprising means for removably attaching the electrodes to a probe.

36. The device of claim 35 wherein the electrodes are disposable.

37. A method of relieving pain comprising the steps of:
locating on the skin of a patient the site of a medical procedure;
placing first and second electrodes on the skin of the patient such that the site of the medical procedure is about between the electrodes;
interconnecting the electrodes with an electric waveform generator; and,
adjusting an electrical output supplied to the electrodes until the patient experiences a gentle tingling sensation from the electrical output.

38. The method of claim 37 wherein the step of adjusting an electrical output supplied to the electrodes until the patient experiences a gentle tingling sensation from the electrical output consists of adjusting the potential difference between the electrodes until the patient experiences a gentle tingling sensation from the electrical output.

39. The method of claim 37 wherein the step of adjusting an electrical output supplied to the electrodes until the patient experiences a gentle tingling sensation from the electrical output consists of adjusting the frequency of the waveform supplied to the electrodes until the patient experiences a gentle tingling sensation from the electrical output.

40. The method of claim 37 wherein the step of adjusting an electrical output supplied to the electrodes until the patient experiences a gentle tingling sensation from the electrical output consists of adjusting the wave shape of the waveform supplied to the electrodes until the patient experiences a gentle tingling sensation from the electrical output.

41. The method of claim 38 further comprising the step of determining a time period during which the waveform generator will be operated prior to penetrating patient tissue, the time period being a function patient feedback.

42. The method of claim 38 further comprising the step of determining a time period during which the waveform generator will be operated prior to penetrating patient tissue, the time period being a function of a tissue conductivity measurement.

43. The method of claim 42 further comprising the steps of:
operating the electric waveform generator during the procedure; and,
adjusting a waveform generator control selected from the group consisting of a voltage control, a frequency control and a wave shape control to relieve patient pain during the procedure.

44. The method of claim 42 further comprising the steps of:
operating the electric waveform generator during the procedure; and,
automatically adjusting the potential difference between the electrodes to maintain a pre-determined level of conductivity of the tissue to relieve patient pain during the procedure.

45. The method of claim 44 further comprising the steps of:
operating the electric waveform generator after the procedure during a post-treatment time period; and,
adjusting a waveform generator control selected from the group consisting of a voltage control, a frequency control and a wave shape control to relieve patient pain during the procedure.

46. The method of claim 44 further comprising the steps of:
operating the electric waveform generator after the procedure during a post-treatment time period; and,
automatically adjusting the potential difference between the electrodes to maintain a pre-determined level of conductivity of the tissue to relieve patient pain during the post-treatment time period.
